(19) United States
(12) Patent Application Publication
(10) Pub. No.: US 2010/0298839 A1
(43) Pub. Date: Nov. 25, 2010

(54) Peelable Sheath and Method of Use

(76) Inventor: Salvatore Castro, Raleigh, NC (US)

Correspondence Address: SYNECOR LLC, P.O. BOX 5325, LARKSPUR, CA, 94977, US

(21) Appl. No.: 12/688,819

(22) Filed: Jan. 15, 2010

Related U.S. Application Data
(60) Provisional application No. 61/144,926, filed on Jan. 15, 2009.

(51) Int. Cl. A61B 17/34; A61B 17/50

(52) U.S. Cl. 606/114; 604/164.05

(57) ABSTRACT

A peelable sheath is used to provide an atraumatic cover for an elongate medical device positionable through an incision. The peelable sheath includes a flexible tube, a pair of tabs on a proximal portion of the flexible tube, and a collar on a proximal portion of the flexible tube. The tabs extend laterally through at least one sidewall opening in the collar. The flexible tube is slidably disposed in a lumen of the collar. In use, the sheath is positioned covering at least a distal portion of the elongate medical device, and the elongate medical device is inserted into an incision or other opening in a patient. The sheath is withdrawn from the elongated medical device by pulling the tabs outwardly relative to the longitudinal axis of the flexible tube, causing at least a proximal portion of the flexible tube to split longitudinally into at least two longitudinal sections. The longitudinal sections move proximally through the at least one sidewall opening as the tabs are pulled.

### RELATED APPLICATIONS

This application claims the benefit of U.S. application Ser. No. 61/144,926, filed Jan. 15, 2009.

### FIELD OF THE INVENTION

The present invention relates generally to the field of sheaths used to facilitate passage of other medical instrument into body cavities or vessels.

### BACKGROUND

In many percutaneous medical procedures tubular sheaths are used as passageways providing access for other medical devices into body cavities or vessels. For example, vascular sheaths are used to give access into the vasculature for catheters, cardiovascular leads, or other therapeutic or diagnostic devices. In some such procedures, the sheath is passed into a blood vessel and the catheter or other device is inserted through the lumen of the sheath into the body. The sheath is next removed from the vessel, leaving the catheter or other device remaining in the vessel. In some cases where the catheter/lead etc has a large hub or connector at its proximal end, the sheath must be longitudinally split or peeled apart to allow it to be removed from the catheter.

In conventional laparoscopic procedures, multiple small incisions or ports are formed through the skin and underlying muscle and peritoneal tissue to provide access to the peritoneal cavity for the various instruments and scopes needed to complete the procedure. Trocar sleeves or sheaths are positioned within the incisions, and the instruments are passed through these sleeves into the cavity. These sleeves or sheaths remain in the incision for the duration of the procedure and are not peeled away from the instruments passed through them.

Applicant's co-pending U.S. application Ser. No. 12/649307, filed Dec. 29, 2009, Attorney Docket No. TRX-1820, ACTIVE INSTRUMENT PORT SYSTEM FOR MINIMALLY-INVASIVE SURGICAL PROCEDURES, which is incorporated herein by reference, describes an active port system used to give multi-instrument access into the abdominal cavity. The system disclosed in the prior application includes an insertion tube through which a plurality of access tubes extend. At least two of the access tubes extend from the distal end of the insertion tube. Link arms coupled to the distal portions of these access tubes are pivotable to change the lateral spacing of the access tubes. In use, the insertion tube is introduced through an incision in the umbilicus or another area of a body wall such as the abdominal wall. Instruments are passed through the plurality of access tubes and used to gain access to an operative site within the body cavity. It is desirable to minimize trauma to surrounding tissue as the distal end of the system (e.g. the distal portions of the access tubes and link arms the insertion tube) is inserted into the abdominal cavity. The present application discloses a sheath suitable for this purpose.

As will be appreciated from the discussion that follows, the disclosed sheath is equally suitable for use other types of access systems, including but not limited to more conventional laparoscopic trocar systems, systems for natural orifice surgical access, and systems providing percutaneous intravascular access.

### BRIEF DESCRIPTION OF THE DRAWINGS

FIG. 1 is a perspective view of a first embodiment of a sheath system;

FIG. 2 is an exploded view of the system of FIG. 1;

FIG. 3A is a perspective view of the funnel of the sheath system of FIG. 1;

FIG. 3B is a side elevation view of the funnel of FIG. 3A;

FIG. 4 is a perspective view of the sheath system of FIG. 1 after peeling;

FIG. 5A a surgical access system which can be incorporate the sheath system of FIG. 1;

FIG. 5B illustrates positioning of the sheath system of FIG. 1 with the surgical access system of FIG. 5A;

FIG. 6 is similar to FIG. 5, but shows the tip removed from the sheath and the sheath having been peeled;

FIG. 7 is similar to FIG. 6, but illustrates advancement of the peeled sheath towards excised tissue to be withdrawn from the body;

FIG. 8 is a perspective view of a second embodiment of a sheath system;

FIG. 9 is an exploded view of the sheath system of FIG. 8.

### DETAILED DESCRIPTION

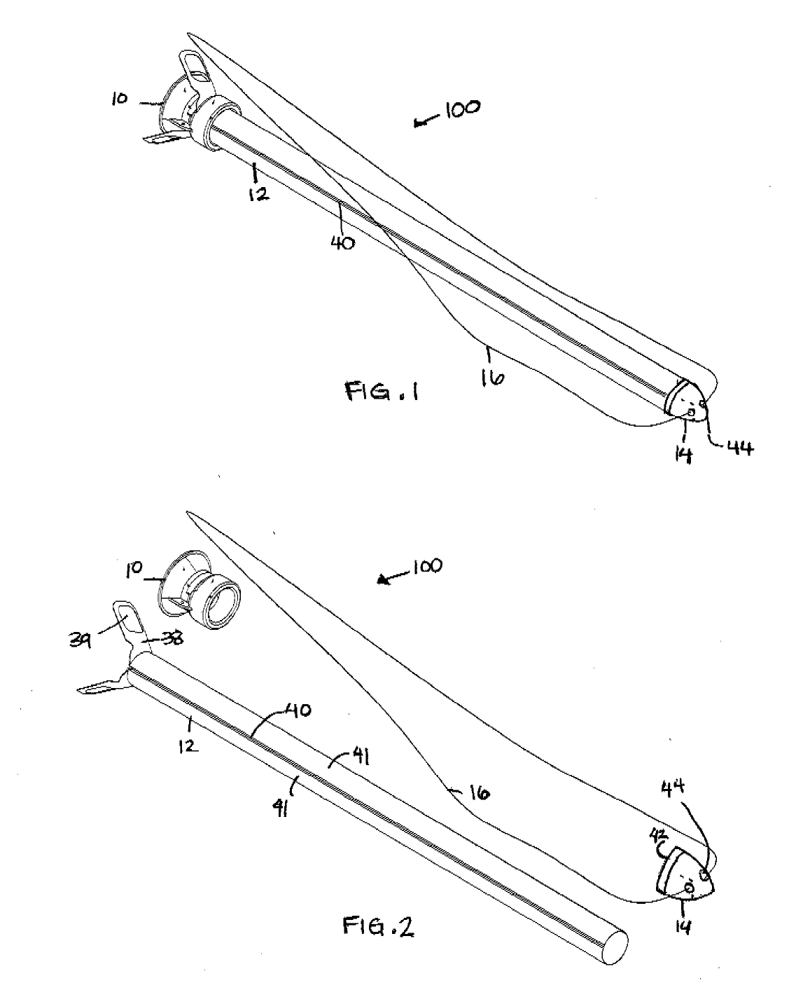

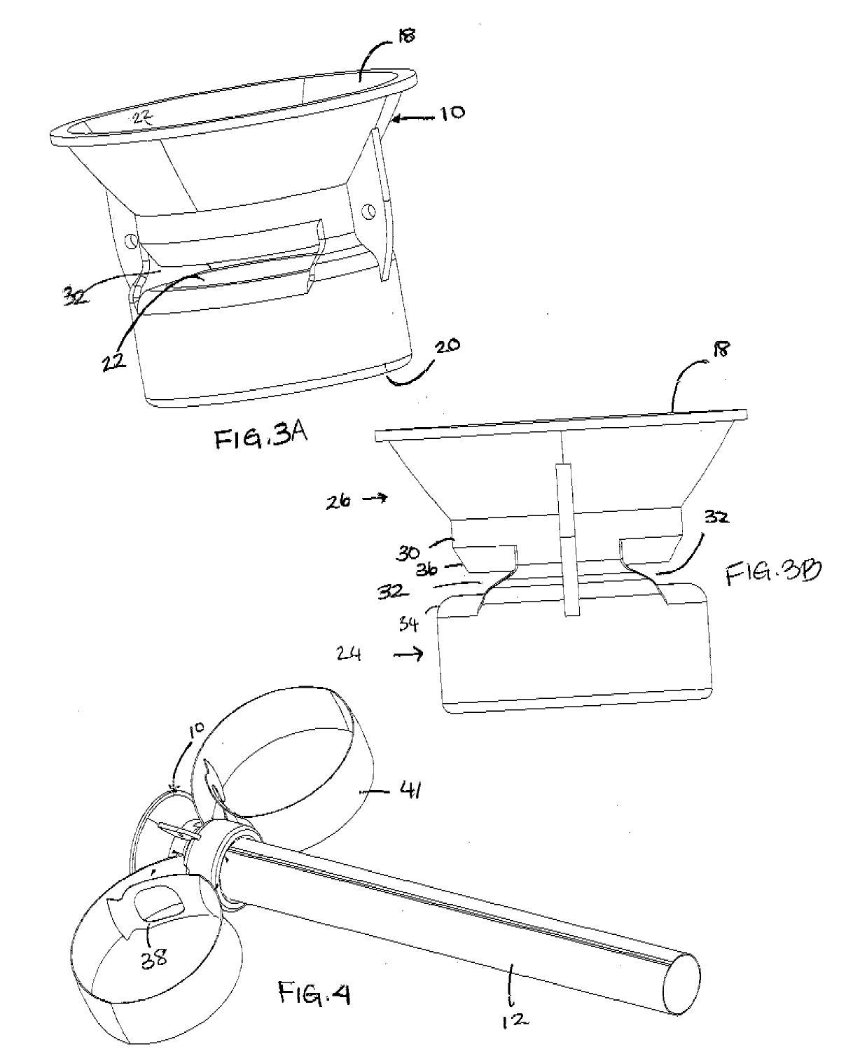

FIGS. 1 and 2 illustrate a first embodiment of a peelable sheath system 100. In general, the system includes a funnel 10, a sheath 12 coupled to the funnel, a tip 14 positioned on the distal end of the sheath 12, and a tether 16 coupled to the tip 14.

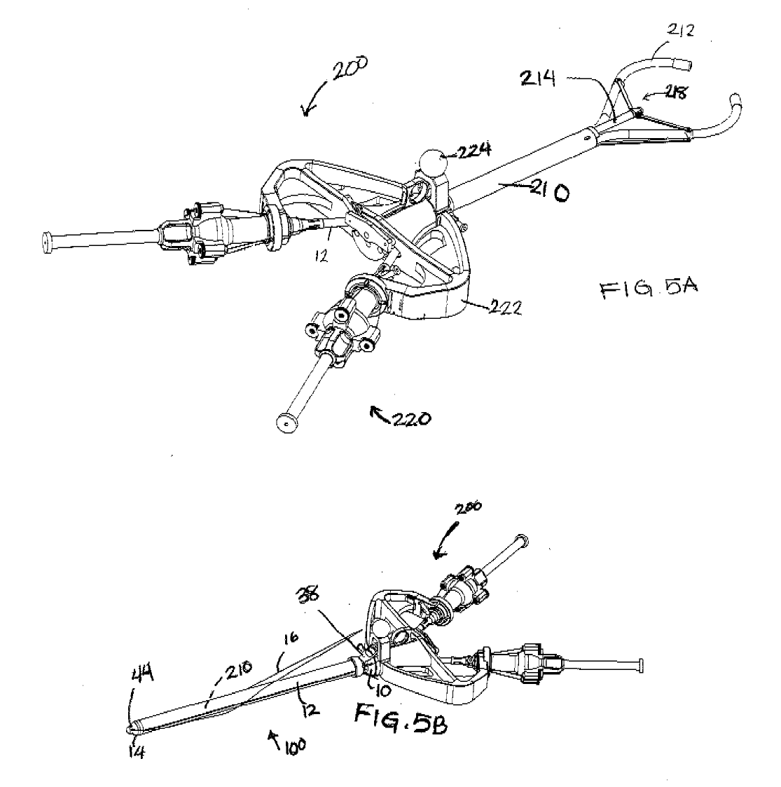

Referring to FIG. 3A, funnel 10 is a tubular element having a proximal opening 18 and a distal opening 20, with a lumen 22 extending between the proximal and distal openings. FIG. 3B best shows that in the illustrated embodiment, the funnel 10 has a generally cylindrical distal section 24, and a proximal section 26 that tapers inwardly from the proximal opening 18 to a cylindrical base 30. The base 30 has a diameter that is smaller than the diameter of the distal section 24.

A pair of slots 32 are formed in the wall of the funnel 10 between the base 30 and the distal section 24. The entrance to each slot 32 has a radiused lower edge 34 and a beveled upper edge 36. The slots 32 extend into the lumen 22 of the funnel 10.

Referring to FIG. 2, the sheath 12 is a tubular sleeve made of PTFE or other suitable material. A pair of finger grips 38 extend angularly from the proximal end of the sheath 12. Each finger grip 38 has an opening 39 through which a user can thread his/her finger to grasp the finger grip 38.

A pair of longitudinal split lines 40 (one is visible) are positioned opposite one another along the body of the sheath and divide the sheath into longitudinal body sections 41. The split lines 40 can extend the full length of the sheath, or they may terminate proximal to the distal end of the sheath. Split lines 38 are formed using any process that will make the split lines 40 weaker than the surrounding material, so that the sheath will preferentially split along the split lines 40 when a user pulls the finger grips 38 away from one another. Suitable processes for forming the split lines 40 include etching or scoring of the sheath material, or manufacturing the split lines 40 using a material less resistant to tearing than the surrounding material.

The tip 14 is a distally tapered element formed of polymeric or other material. The tether 16 is attached to the tip. In one embodiment, the tether 16 extends through openings 44 formed in the tip as shown. The tip 14 is coupled to the distal end of sheath 12, and is removed from the sheath when a user pulls on the tether 16.

Various arrangements may be used to couple the tip 14 to the sheath 12. In one embodiment, the proximal end of the tip has an opening 42 that is press fit over the distal end of the sheath. Alternatively, a proximal portion of the tip may be inserted into the lumen of the sheath. In either case, friction or light adhesives may be used to retain the tip on the sheath until it is actively removed using the tether.

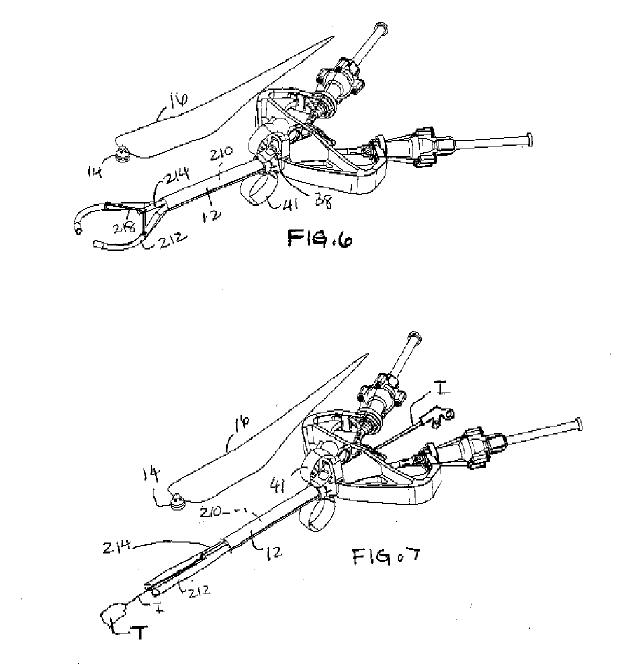

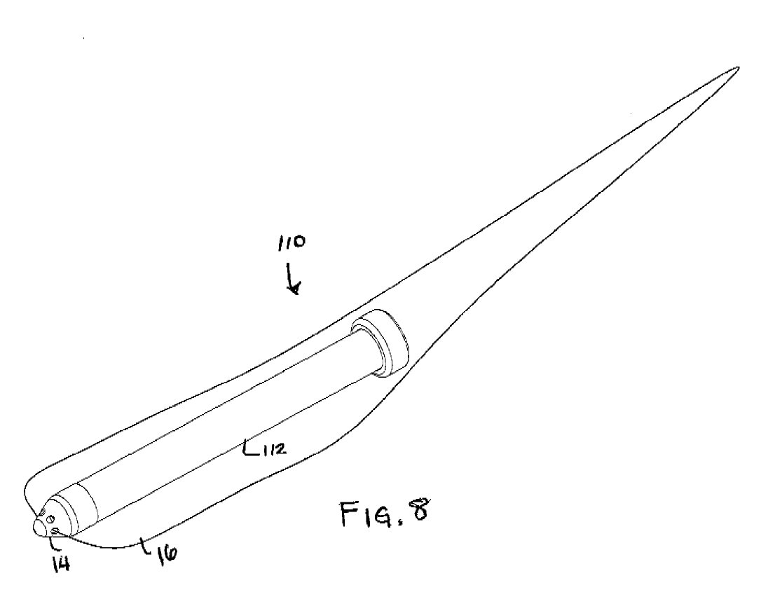

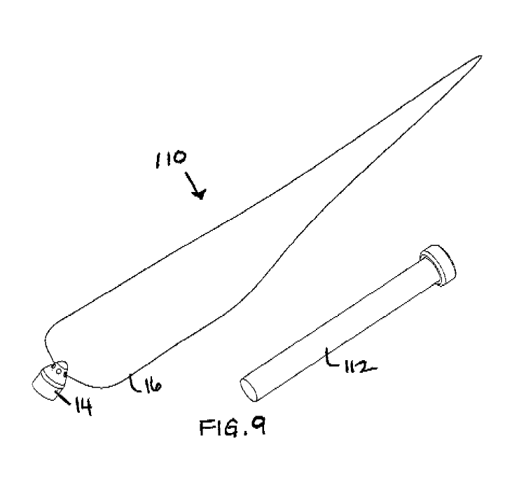

As shown in FIG. 1, the sheath 12 and funnel 10 are assembled such that the proximal portion of the sheath 12 is positioned within the lumen 22 of the funnel 10. The finger grips 38 extend through the slots 32 in the funnel 10. When a user engages the finger grips 38 and pulls them laterally away from the longitudinal axis of the sheath, the sheath separates along the split lines 40, allowing the user to pull the separated longitudinal body sections 41 through the slots 32 in the funnel 10 as shown in FIG. 4. By pulling the proximal portions of the body sections 41 through the slots 32, the user retracts the distal end of the sheath proximally. If desired, the user can split the sheath along its entire length and then fully withdraw the longitudinal body sections 41 from the funnel 10.

Use of the system 100 will next be described with reference to an access system 200 of the type disclosed in Applicant's U.S. Provisional Application No. 61/141088, filed Dec. 28, 2008 (Attachment A hereto). Referring to FIG. 5A, access system 200 includes an insertion tube 210 through which a plurality of access tubes 212, 214 extend. In use, the insertion tube extends through an incision in the umbilicus or another area of a body wall such as the abdominal wall. Instruments are passed through the plurality of access tubes and used to gain access to an operative site within the body cavity. The plurality of access tubes includes a pair of flexible tubes 212 having distal ends that may be articulated using proximal actuators 220. A deployment system including link arms 218 is positioned to move the flexible tubes 212 between a streamlined orientation (similar to the position shown in FIG. 7) for insertion into a body cavity and the spaced apart position shown in FIG. 5A. The system components are supported by a frame 222 and include a coupling 224 that mounts the frame to a fixture within the operating room.

Referring to FIG. 5B, when the sheath system 100 is used in conjunction with the access system 200, the funnel 10 is disposed around a proximal portion of the insertion tube 210, and the sheath 12 covers at least a portion of the insertion tube, as well as the flexible tubes 212 and link arms 218 (not visible in FIG. 5B). The tip 14 is coupled to the distal end of the sheath 12, and the tether 16 extends proximally. To accommodate the sheath 12 and tip 14, the flexible tubes 212 and link arms 218 are positioned in the streamlined orientation.

The distal end of the access system 200, with the sheath system 100 thereon as in FIG. 5B, is inserted into the incision in the patient. The finger grips 38 of the sheath 12 and the tether 16 coupled to the tip 14 extend out of the body. If insufflation gas is being introduced into the body cavity through the insertion tube 210 or associated tubes, the holes 44 in the tip 14 allow the gas to pass into the body cavity. It should be noted that if an endoscope is to be positioned within the insertion tube or one of the associated access tubes for visualization during insertion of the system into the body, an optically transparent tip 14 may be used.

After the insertion tube 210 has been advanced into the body, the user pulls on the tether 16 to detach the tip 14 from the distal end of the sheath 12. The tip hangs freely within the body cavity, suspended from the tether. The user may couple a proximal portion of the tether 16 to the funnel 10 or to a portion of the access system 200 that remains outside the body.

Next, the user retracts the sheath 12 to allow the tubes 212 and link arms 218 be exposed so they can deployed to an expanded position. To retract the sheath, the user pulls the finger grips 38 apart as discussed in connection with FIG. 4, thus peeling apart the longitudinal sections 41 of the sheath and causing the distal end of the sheath to slide proximally on the insertion tube 210. Once the sheath has been retracted sufficiently to uncover the tubes 212 and link arms 218, the link arms 218 may be actuated to deploy the tubes 212 to the position shown in FIG. 6.

The sheath system 100 provides the user with a number of options concerning how to proceed at this point. The user can fully separate the longitudinal sections 41 of the sheath, and remove the longitudinal sections 41 from the funnel 10, leaving only the funnel in place surrounding the insertion tube 210. Once the user has finished using the access system 200, s/he can withdraw access system 200 from the body, thereby removing the insertion tube 210 from the funnel 10 and leaving the funnel in place within the body incision. The funnel can thus serve as an access port through the incision, allowing other instruments or instrument systems to be used through the funnel 10.

In an alternate procedure, rather than fully separating the sheath, the more distal portion of the sheath can remain intact as shown in FIG. 6. If the user selects this method, the separated longitudinal sections 41 extend outside of the body and the intact distal portion of the sheath is disposed on the insertion tube 210. The sheath 12 can remain in this position until the sheath system 100 and access system 200 are removed from the body. Alternatively, the distal portion of the sheath can be later used to cover or contain tissue extracted from the body cavity during the procedure. For example, FIG. 7 shows a mass of tissue T engaged by an instrument I passed through tube 214. In this figure, the tubes 212 and links 218 have already been moved back into the streamlined position for removal from the body. The user can choose to advance the intact distal portion of the sheath over the mass of tissue T by pushing the peeled longitudinal sections 41 of the sheath distally through the slots in the funnel 10. If more complete containment of the tissue is desired, the tether 16 is manipulated to reposition the tip 14 on the distal end of the sheath. Once the tissue is contained, the systems 100 and 200 are removed from the body together.

A second embodiment of a sheath system 110 is shown in FIGS. 8 and 9. The second embodiment differs from the first embodiment in that in the second sheath system 110, the sheath 112 is not a peelable sheath. In this embodiment, the sheath may be made of polyolefin or other suitable material. During use of the sheath 112 with the access system 200 shown in FIGS. 5A-7, the sheath 112 is positioned covering the tubes 212 and links 218, and the tip 14 is positioned on the distal end of the sheath 112 as described above. After the insertion tube 210 of the system 200 has been inserted into the body, the tip is detached using the tether 16 as described above. The sheath 112 is manually retracted in a proximal direction to expose the distal ends of the tubes 212 and links 218 and remains in place over the insertion tube 210 throughout the duration of the procedure. If desired, the sheath 112 may be distally advanced at a later stage for use in containing tissue to be removed from the body.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed.

Any and all patents, patent applications and printed publications referred to above are incorporated by reference.

I claim:

1. A peelable sheath for a medical device, the sheath comprising:
a flexible tube having a longitudinal axis and a pair of longitudinal separation lines;
a pair of tabs on a proximal portion of the flexible tube, the tabs positioned such that the pulling the tabs outwardly relative to the longitudinal axis causes the tube to split along the separation lines; and
a collar having a lumen and at least one sidewall opening, the collar positioned on a proximal portion of the tube such that the tube is slidably disposed in the lumen and such that the tabs extend through the at least one sidewall opening.

2. The peelable sheath of claim 1, wherein the longitudinal separation lines define longitudinal sections of the flexible tube, and wherein the at least one sidewall opening is positioned such that pulling the tabs outwardly relative to the longitudinal axis causes the separated proximal portions of the longitudinal sections to move proximally through the at least one sidewall opening.

3. The peelable sheath of claim 2, wherein the collar includes a pair of sidewall openings, each tab extending through a corresponding one of the sidewall openings such that pulling the tabs outwardly relative to the longitudinal axis causes the separated proximal portion of each of the longitudinal sections to move proximally through a corresponding one of the sidewall openings.

4. The peelable sheath of claim 1, wherein at least a portion of the collar forms a funnel around the lumen.

5. The peelable sheath of claim 1, wherein the flexible tube includes a distal end, and wherein the sheath further includes a removable cap on the distal end.

6. The peelable sheath of claim 5, wherein the cap includes a distal taper.

7. The peelable sheath of claim 5, wherein the cap includes a plurality of openings.

8. The peelable sheath of claim 5, further including a tether coupled to the distal cap, the tether retractable to detach the cap from the distal end of the flexible tube.

9. The peelable sheath of claim 5 wherein the cap is visually transparent.

10. An access system for surgical procedures, the access system comprising:
an insertion tube;
a pair of flexible instrument delivery tubes extending through the insertion tube;
peelable sheath positioned over the insertion tube and the flexible instrument delivery tubes, the peelable sheath including
a flexible tube having a longitudinal axis and a pair of longitudinal separation lines,
a pair of tabs on a proximal portion of the flexible tube, the tabs positioned such that the tube may be divided along the separation lines by pulling the tabs outwardly relative to the longitudinal axis, and
a collar having a lumen and at least one sidewall opening, the collar positioned on a proximal portion of the tube such that the tube is disposed in the lumen and such that the tabs extend through the at least one sidewall opening.

11. A method of using a peelable sheath, comprising:
positioning at least a distal portion of an elongate medical device within a peelable sheath, the peelable sheath comprising a flexible tube, a pair of tabs on a proximal portion of the flexible tube, and a collar on a proximal portion of the flexible tube wherein the tabs extend laterally through at least one sidewall opening in the collar and wherein the flexible tube is slidably disposed in a lumen of the collar;
inserting the distal portion of the elongate medical device, with the sheath thereon, into an opening in a patient;
pulling the tabs outwardly relative to the longitudinal axis of the flexible tube, causing at least a proximal portion of the flexible tube to split longitudinally into at least two longitudinal sections, the longitudinal sections moving proximally through the at least one sidewall opening as the tabs are pulled.

12. The method of claim 11, wherein pulling the tabs retracts the distal end of the flexible tube in a proximal direction.

13. The method of claim 12, wherein pulling the tabs causes the flexible tube to split from the proximal end to the distal end of the flexible tube.

14. The method of claim 13, further including withdrawing the split distal ends of the longitudinal sections through the sidewall opening to remove the flexible tube from the collar.

15. The method of claim 14, wherein the method includes, after removing the flexible tube from the collar, leaving a distal portion of the collar positioned within the opening in the patient.

16. The method of claim 15, further including, with the distal portion of the collar positioned within the opening, removing the elongate medical device from the collar, and inserting a second elongate medical device into the lumen of the collar.

17. The method of claim 11, wherein the peelable sheath includes a removable cap on its distal end, and wherein the method includes detaching the removable cap from the distal end.

18. The method of claim 17, wherein detaching the removable cap includes pulling a tether attached to the removable cap.

19. The method of claim 12, further including:
with the distal end of the flexible tube in a retracted position on the elongate medical device, performing a procedure within the patient, said procedure including engaging a mass of tissue at the distal end of the elongate medical instrument;
advancing the distal end of the flexible tube distally to advance the distal end over the mass of tissue; and
withdrawing the flexible tube, with the mass of tissue at least partially contained in its distal end, through the opening and out of the patient.

20. The method of claim 19, further including:
prior to the withdrawing step, positioning a cover over the distal end of the flexible tube to contain the tissue mass therein.
